(19) United States
(12) Patent Application Publication
(10) Pub. No.: US 2026/0263334 A1
(43) Pub. Date: Sep. 10, 2026

(54) COMPOSITIONS CONTAINING NITRATE-RICH FOODS AND EXTRACTS FOR SHIFTING THE ORAL MICROBIOME

(71) Applicant: WM. WRIGLEY JR. COMPANY, Chicago, IL (US)

(72) Inventors: Minmin TIAN, Chicago, IL (US); Michael DODDS, Chicago, IL (US)

(21) Appl. No.: 19/167,638

(22) PCT Filed: Mar. 22, 2024
(86) PCT No.: PCT/US2024/021037

§ 371 Date: Sep. 22, 2025

Related U.S. Application Data
(60) Provisional application No. 63/454,457, filed on Mar. 24, 2023.

(51) Int. Cl. A61K 8/19; A23L 29/00; A61K 8/02; A61K 8/9789; A61K 8/9794; A61Q 11/00

(52) U.S. Cl. CPC A61K 8/19; A61K 8/0216; A61K 8/9789; A61K 8/9794; A61Q 11/00; A23L 29/015; A61K 2800/92

(57) ABSTRACT

The present disclosure is directed to oral and/or confectionary compositions comprising an effective amount of a nitrate, for example, a dietary nitrate derived from a nitrate-rich food and/or a nitrate-rich extract, for use in reducing volatile sulfur compounds (VSCs) in the oral cavity, and freshening breath.

### CROSS REFERENCE TO RELATED APPLICATION(S)

This application claims the benefit of priority to U.S. Provisional Application Ser. No. 63/454,457, which was filed Mar. 24, 2023, the disclosure of which is hereby incorporated by reference as if set forth in its entirety.

### BACKGROUND OF THE DISCLOSURE

The present disclosure is related to oral compositions comprising nitrates and use thereof. In particular, the present disclosure is related to confectionary compositions comprising sodium or potassium nitrate, and dietary nitrates derived from nitrate-rich foods and/or nitrate-rich extracts that, when consumed, produce a breath freshening effect while simultaneously exhibiting oral care effects against volatile sulfur compounds (VSCs), and enhancing heart health.

Oral malodor or “halitosis” used herein relates to bad breath caused by physiologic (transient or temporary) and/or pathologic conditions. Physiological causes of halitosis include: halitosis caused by deleterious habits, morning breath, and xerostomia (dry mouth). Pathological causes for halitosis include: secondary or oral tissue conditions associated with gingival and periodontal diseases, acute necrotizing ulcerative gingivitis, residual post-operative blood, debris under dental appliances, ulcerative lesions of the oral cavity, coated tongue, xerostomia, salivary gland diseases and Tonsilloliths (tonsil stones).

Oral malodors are produced mainly due to the breakdown of proteins into individual amino acids, followed by the further breakdown of certain amino acids to produce detectable foul gases. The oral cavity provides a positive growth environment for gram-negative anaerobes that metabolize proteins as an energy source via breakdown of proteinaceous substrates from impacted food particles and sloughed off oral cellular debris. Often, inflammation causes these cells to be shed at a faster rate than the saliva can cleanse. Bacterial action then hydrolyzes the proteins to amino acids; and the amino acids that contain sulfur functional groups, e.g., methionine and cysteine, serve as precursors to VSCs.

These gaseous VSCs are responsible for oral malodor, and consist primarily of hydrogen sulfide (H₂S), methyl mercaptan (CH₃SH) and dimethyl sulfide [(CH₃)₂S]. For example, amino acids methionine and cysteine are reduced to hydrogen sulfide and methyl mercaptan, respectively, in the presence of sulfhydrase-positive microbes. Methyl mercaptan has been found to be the main component of tongue dorsal surface malodor in patients with periodontal disease, whereas hydrogen sulfide predominates in orally healthy subjects.

Although bad breath primarily represents a source of embarrassment or annoyance, the VSCs most responsible for halitosis are also potentially damaging to the tissues in the mouth, and can lead to periodontitis (inflammation of the gums and ligaments supporting the teeth). In particular, VSCs have been found to damage the collagen and proteoglycan components in connective tissue by cleaving disulfide bonds. This de-aggregation of the extracellular matrix allows microbes to permeate the oral mucosa. As bacteria further accumulates in pockets that form next to the teeth, periodontal disease progresses, as well as halitosis. If the periodontal disease advances significantly, overall systemic health may be jeopardized; for example, periodontal bacterial by-products can enter the blood stream and may result in heart disease, stroke and under-weight babies at birth.

For the treatment of oral malodor, the public has increasingly turned to commercially available mouth-freshening products. The market for these products has been growing continuously as sufferers from chronic oral malodor experience personal discomfort and social embarrassment.

Various compounds such as chlorine dioxide, sodium chlorite, and metal salts such as zinc and copper have been used as VSC neutralizing agents in a variety of oral compositions. Such compounds have been provided and are available today as mouthwashes and rinses for the prevention and/or treatment of oral malodor. In general, many of the oral rinses available today are used for the prevention and/or treatment of oral malodor or halitosis, but for chronic bad breath, many rinses offer little to no help.

While being effective in fighting oral malodor, compounds such as chlorine dioxide, sodium chlorite, and metal salts such as zinc and copper impart strong, unpleasant flavors and aromas thereby negatively impacting taste and deterring use. Often, mouth rinses incorporating these compounds can also cause some generalized irritations to the oral cavity such as desquamation, ulceration, and inflammation. Moreover, oral compositions incorporating these compounds (other than mouthwashes and rinses) which are retained in the mouth for longer periods of time, such as chewing gums, mints, and lozenges, further enhance irritations to the oral cavity when these compounds are employed.

Recent studies have shown that anti-septic mouthwash treatment kills not only harmful oral bacteria but also benign microorganisms and disrupts oral microbiota. For example, mouthwash containing 0.12% of chlorhexidine reduced both VSC-producing bacteria and enteric nitric oxide producing bacteria. It reduced both oral and plasma nitrite levels in healthy human volunteers, and was associated with a sustained increase in systolic and diastolic blood pressure. The study suggested that oral nitrate reducing bacteria may contribute to host nitrite and thus NO levels, with measurable physiological effects. Anti-bacterial compounds including chlorhexidine, chlorine dioxide, sodium chlorite, etc., may severely suppress oral microflora and affect systemic nitrite levels and hence blood pressure in healthy volunteers.

In view of the preceding challenges, there is a continued need to develop an effective treatment for oral malodor caused by physiologic and/or pathologic conditions.

### SUMMARY OF THE DISCLOSURE

In one aspect, the present disclosure is related to an oral or confectionary composition comprising an effective amount of a nitrate for reducing volatile sulfur compounds in the oral cavity.

In another aspect, the present disclosure is related to an oral composition comprising an effective amount of a nitrate for reducing volatile sulfur compounds in the oral cavity; and an effective amount of ascorbic acid, or a suitable salt thereof.

In a further aspect, the present disclosure is related to a method of freshening breath comprising consuming a confectionary composition comprising an effective amount of a nitrate.

In some embodiments of the preceding aspects, the nitrate comprises sodium nitrate or potassium nitrate.

In some embodiments of the preceding aspects, which may be combined with any of the preceding embodiments, the nitrate comprises a dietary nitrate derived from a nitrate-rich food, a nitrate-rich extract, or combinations thereof.

In some embodiments of the preceding aspects, which may be combined with any of the preceding embodiments, the nitrate is present in an amount of about 0.01% to about 5% by weight of the confectionary composition.

In some embodiments of the preceding aspects, which may be combined with any of the preceding embodiments, the nitrate-rich food is selected from beetroot, kale, arugula, chard, spinach, parsley, watercress, fennel, Chinese cabbage, bok choy, leek, lettuce, celery, radish, turnip, rocket, beet greens, mustard greens, carrots, onions, garlic, kohlrabi, chicory leaf, bean sprout, watermelon, kiwi, apples, pomegranate, bananas, oranges, strawberries, peaches, pears, grapes, chocolate, or combinations thereof.

In some embodiments of the preceding aspects, which may be combined with any of the preceding embodiments, the nitrate-rich extract is selected from beetroot extract, celery extract, or combinations thereof.

In some embodiments of the preceding aspects, which may be combined with any of the preceding embodiments, the confectionary composition is in the form of a breath mint, low boiled candy, chewing gum, chewy candy, hard boiled candy, coated candy, lozenge, syrup, pressed mint, throat drop, or chocolate.

In some embodiments of the preceding aspects, which may be combined with any of the preceding embodiments, the chewing gum is a sugar-free chewing gum.

In some embodiments of the preceding aspects, which may be combined with any of the preceding embodiments, the confectionary composition further comprises ascorbic acid, or a suitable salt thereof.

In some embodiments of the preceding aspects, which may be combined with any of the preceding embodiments, the ascorbic acid, or a suitable salt thereof, is present in an amount of about 0.01% to about 5% by weight of the confectionary composition.

### BRIEF DESCRIPTION OF THE DRAWINGS

FIG. 1 shows an exemplary calibration curve produced by the nitrate/nitrite test assay based on the modified Griess test disclosed herein.

FIG. 2 shows the ex vivo production of H₂S by saliva mixed with nitrate, saliva mixed with cysteine, and saliva mixed with a combination of cysteine and nitrate.

FIG. 3 shows the ex vivo production of nitrite by saliva alone, saliva mixed with cysteine, saliva mixed with nitrate, and saliva mixed with a combination of nitrate and cysteine.

FIGS. 4A and 4B show the ex vivo production of nitrite from the saliva of two subjects when the saliva was mixed with cysteine and/or nitrate, as compared to saliva alone, or control mixtures containing water, nitrate and cysteine.

FIG. 5 shows the activity of nitrate reductase after chlorhexidine (CHX) treatment of Aggregatibacter actinomycetemcomitans (A.a.).

FIG. 6 shows the activity of nitrate reductase after CHX treatment of Veillonella atypica (V.a.).

FIG. 7 shows the activity of nitrate reductase after treatment of A.a. with magnolia bark extract (MBE).

FIG. 8 shows the activity of nitrate reductase after MBE treatment of V.a.

### DETAILED DESCRIPTION OF THE DISCLOSURE

The present disclosure is related to an oral composition comprising an effective amount of a nitrate and methods of use thereof.

Nitrates, for example, sodium or potassium nitrate, and dietary nitrates derived from nitrate-rich foods and extracts, can be actively taken up by salivary fluid. Salivary nitrate is metabolized to nitrite by nitrate reductases produced by anaerobic oral bacteria under anaerobic conditions. Nitrite serves as the precursor for nitric oxide which has been linked to various health benefits, such as a reduction in blood pressure and overall maintenance of cardiovascular and systemic health.

Surprisingly, it has been found that, in the absence of nitrates, oral bacteria utilize sulfur-bearing proteins as a primary food source and break these down to generate volatile sulfuric compounds (VSCs) that are responsible for halitosis. It has further been found that, in the presence of nitrate-rich foods, oral bacteria preferentially utilize nitrate as the food source and generate nitric oxide, which has been shown to reduce blood pressure, and benefit cardiovascular and systemic health. As a result, less VSCs are generated which are responsible for oral malodor and damage to soft and hard tissues of the teeth.

Without being bound by theory, when dietary nitrates are added into oral compositions, such as confectionary compositions, they can help shift the oral microbiome and significantly reduce the production of VSC, thereby achieving a breath freshening effect. They may further be converted to nitric oxide, thereby helping to decrease systolic and diastolic blood pressure for enhanced heart health.

In various aspects, the present disclosure provides for an oral composition comprising a nitrate present in an effective amount for reducing volatile sulfur compounds in the oral cavity of a user. In various aspects, the oral composition may be in any form suitable for application to an oral surface of humans, dogs, cats or other animals and provides either a cosmetic prophylactic or therapeutic benefit within or derived from the oral cavity. In various aspects, the oral composition may include a dentifrice such as a powder or paste; an edible film or bioadhesive film; a confectionary composition including but not limited to breath mints, low boiled candy, chewing gum, such as sugar-free chewing gum, chewy candy, hard boiled candy, coated candy, lozenges, syrups, pressed mints, throat drops, and chocolates; pet foods, chews or biscuits and the like. In certain embodiments, the consuming or masticating of the oral composition may be repeated at regular intervals.

As used herein, the term “effective amount” refers to the level, amount, serving, or precent which produces or is capable of producing a desired effect. All percentages and ratios used herein are by weight of the total composition.

In various aspects, suitable nitrates that may be included in the oral composition of the disclosure may be derived from any source of nitrate ions, including, but not limited to, nitrate salts, such as sodium nitrate or potassium nitrate; or dietary nitrates derived from nitrate-rich foods and/or extracts. In various aspects, the nitrates may be derived from nitrate-rich foods including, but not limited to, beetroot, kale, arugula, chard, spinach, parsley, watercress, fennel, Chinese cabbage, bok choy, leek, lettuce, celery, radish, turnip, rocket, beet greens, mustard greens, carrots, onions, garlic, kohlrabi, chicory leaf, bean sprout, watermelon, kiwi, apples, pomegranate, bananas, oranges, strawberries, peaches, pears, grapes, chocolate, or combinations thereof. In other aspects, the nitrates may be derived from nitrate-rich extracts including, but not limited to, beetroot extract, celery extract, or combinations thereof.

In another aspect, the present disclosure provides an oral composition comprising a nitrate present in an effective amount for reducing volatile sulfur compounds in the oral cavity of a user and an effective amount of ascorbic acid, or a suitable salt thereof. Without being bound by theory, by combining the nitrates with ascorbic acid, the risk of formation of potentially carcinogenic nitrosamines may be reduced.

In various aspects, the nitrate may be present in the oral composition in any amount that is effective for reducing volatile sulfur compounds in the oral cavity. In some aspects, the nitrate is present in an amount of about 0.01% to about 5% by weight of the oral composition, or about 0.01%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or 5% by weight of the oral composition, or about 0.1% to about 3% by weight of the oral composition, or about 1% to about 5% by weight of the oral composition, or about 3% to about 5% by weight of the oral composition, or any percentage between any of these values. In further aspects, the ascorbic acid or suitable salt thereof may be present in the oral composition in any amount that is effective for reducing the risk of formation of nitrosamines. In some aspects, the ascorbic acid, or suitable salt thereof is present in an amount of about 0.01% to about 5% by weight of the oral composition, or about 0.01%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or 5% by weight of the oral composition, or about 0.1% to about 3% by weight of the oral composition, or about 1% to about 5% by weight of the oral composition, or any percentage between any of these values.

In various aspects, the oral compositions of the disclosure contain an effective VSC reducing amount of a nitrate combined with a suitable carrier. A suitable carrier may be a food-acceptable or food contact acceptable material in which the nitrate may be incorporated or dispersed without adverse effect. A suitable carrier may include a water-soluble solid or chewable solid such as a confectionery composition. Another suitable carrier may be a dentifrice such as a paste or powder. Other suitable carriers for cats, dogs and other animals include but are not limited to chews, biscuits, kibble (dry), and canned (wet/soft) pet foods.

The term “confectionery composition” as used herein includes chewing gums, and orally soluble tablets, beads and lozenges. Saliva dissolves the lozenge or chewable gum product, and promotes prolonged contact with oral surfaces so that the delivery of the VSC reducing agents in a lozenge tablet, bead or chewing gum form ensures that an adequate dosage of the active ingredients are delivered to the oral surface when the product is used. Or, the confectionery composition may be in the form of a coating, shell, film, syrup or suspension.

In one aspect, the oral composition of the disclosure may be a chewing gum composition which is suitable for chewing and which comprises 2% or greater of elastomer by weight of the composition. In general, chewing gum compositions are chewed or masticated by consumers, the process by which food is mashed and crushed by teeth. Such chewing gum compositions can take a variety of shapes and forms, for example, a pellet, a gumball, a square, a stick, etc., and may be coated by a variety of materials including but not limited to sugars, polyols, chocolates, syrups, films, and the like, alone or in any combination. Natural or artificial colors and combinations thereof, high intensity sweeteners and flavors may also be added to the coating solution. For pellet or coated chewing gums, zinc salts may be incorporated in a coating or in a center.

A suitable chewing gum may include a sugarless chewing gum comprising an effective VSC reducing amount of a nitrate. Chewing gum formulations may contain, in addition to, a chewing gum base, one or more plasticizing agents, at least one sweetening agent and at least one flavoring agent.

In one aspect, a chewing gum is provided comprising a nitrate in an amount of from about 0.01% to about 5% by weight of the chewing gum, or about 0.01%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or 5% by weight of the chewing gum, or about 0.1% to about 3% by weight of the chewing gum, or about 1% to about 5% by weight of the chewing gum, or any percentage between any of these values.

In another aspect, a mint is provided comprising a nitrate in an amount of from about 0.05% to about 2% by weight of the mint, or about 0.05%, 0.1%, 0.2%, 0.3%, 0.4%, 0.5%, 0.6%, 0.7%, 0.8%, 0.9%, 1%, 1.5%, or 2% by weight of the mint, or about 0.1% to about 1% by weight of the mint, or about 0.5% to about 2% by weight of the mint, or any percentage between any of these values.

In various aspects, an optional coating may also be applied to any of the oral compositions disclosed herein. Coating material appreciated by those skilled in the art may include, but are not limited to waxes, shellac, polyols, carboxymethyl cellulose, polyethylene/malic anhydride copolymer or kappa-carrageenan.

In another aspect, the present disclosure provides for a method for freshening breath comprising consuming the oral or confectionary compositions of the disclosure.

In various aspects, the method may involve consuming an oral composition comprising a nitrate present in an effective amount for reducing volatile sulfur compounds in the oral cavity of a user. In some aspects, the method may involve consuming an oral composition comprising (1) a nitrate present in an effective amount for reducing volatile sulfur compounds in the oral cavity of a user, and (2) an effective amount of ascorbic acid, or a suitable salt thereof. In other aspects, the method may involve consuming a confectionary composition comprising a nitrate present in an effective amount for reducing volatile sulfur compounds in the oral cavity of a user.

In various aspects, the method may involve consuming an oral composition that is present in any form suitable for application to an oral surface of humans, dogs, cats or other animals and provides either a cosmetic prophylactic or therapeutic benefit within or derived from the oral cavity.

### EXAMPLES

The presently disclosed subject matter will be better understood by reference to the following Examples, which are provided as exemplary of the invention, and not by way of limitation.

### Example 1: Nitrate/Nitrite Assay of Dietary Vegetables

Given the importance of dietary intake of nitrates, a number of analytical methods have been developed and published. To date, several commercial testing kits are available for detection of both nitrate and nitrite compounds based on Griess test and ELISA kit. The test allows routine analysis of nitrate/nitrite in human blood, in air and water, and in the environmental soil. In order to perform a quick and accurate analysis of nitrate and nitrite concentrations in foods and in saliva, a nitrate/nitrite test method based on the modified Griess test was developed. This method was able to detect either nitrate or nitrite concentrations from any food extracts and saliva within a minute with a detection limit of 0.1 ppm. It was found that many nitrate-rich foods contained 0.1%-1% of nitrate.

Detailed testing procedures are provided below:

### Step 1. Preparation of a Calibration Curve for Potassium Nitrite

1. 0.5 g of modified Griess reagent (mixture of 0.2% N-(1-naphthyl)ethylenediamine dihydrochloride, and 2% sulfanilamide in 5% phosphoric acid and inert carrier) was dissolved in 100 mL of D.I. water to prepare 0.5% of Griess working solution.
2. 1 g of KNO₂ was dissolved in 100 mL of D.I. water to prepare 1.0% of KNO₂ solution. The solution was further diluted by 100× water to prepare 0.01% of KNO₂ solution.
3. 1 mL of 0.5% modified Griess reagent made from procedure 1 was added and mixed with 0, 5, 10, 15 and 20 μL of 100 ppm (0.01%) KNO₂ solution from procedure 2 to prepare a serial of calibration solutions containing 0, 0.5, 1, 1.5 and 2 ppm of KNO₂.

4. Each vial was briefly vortexed and the absorbance at 540 nm was measured by a spectrophotometer.

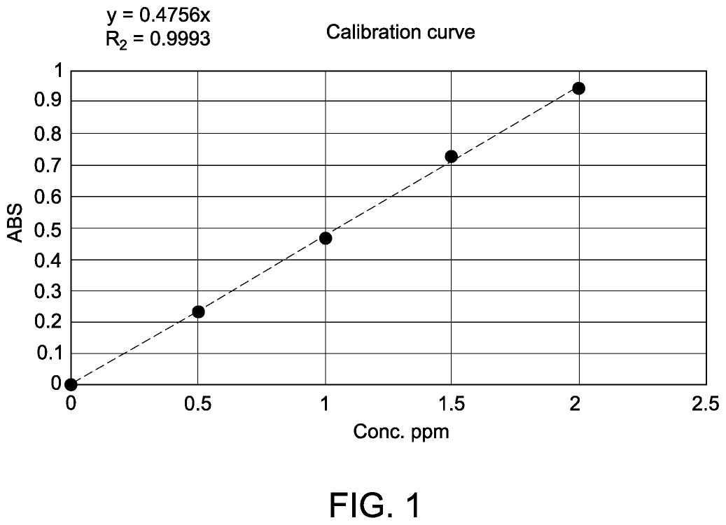

The resulting calibration curve is shown in FIG. 1.

Step 2. Measurement of Salivary Nitrate Level, and after Addition of KNO₃.

5 mL of stimulated or unstimulated saliva was collected and 5 mL of D.I. water was added for a 50% dilution. The diluted saliva was vortexed briefly, and 0.05 mL of 50% salivary solution was pipetted to 1 mL of 0.5% Griess working solution (Step-1 above). The solution was shaken briefly and the absorbance at 540 nm was measured by a spectrophotometer. The solution served as a representation of the background NO 2 level in saliva.

Step 3. Measurement of Salivary of NO⁻₂ Production
1. 0.5% L-cysteine solution was prepared by dissolving 0.5 g L-cysteine in 100 mL of D.I. water.
2. 0.5 mL of 50% diluted saliva and 0.5 mL of D.I. water were mixed in a 5 mL vial. 0.1 mL of 30% KNO₃ was added and the resulting solution was vortexed. A 1 mL solution containing 25% saliva and 3% of KNO₃ (Solution A) was prepared.
3. 0.5 mL of 50% diluted saliva and 0.5 mL of 0.5% cysteine were pipetted to a 5 mL vial. A 1 mL solution containing 25% saliva and 0.25% L-cysteine (Solution B) was prepared.
4. 0.5 ml of 50% diluted saliva, 0.5 mL of 0.5% cysteine and 0.1 mL of 30% KNO₃ were mixed and vortexed. A 1 mL solution containing 25% diluted saliva, 0.25% L-cysteine and 3% of KNO₃ (Solution C) was prepared.
5. 0.5 ml of D.I. water, 0.5 mL of 0.5% cysteine and 0.1 mL of 30% KNO₃ were pipetted and vortexed. A 1 mL solution containing 0.25% L-cysteine and 3% of KNO₃ (Solution D) was prepared.
6. Every 15 minutes, solution A, B, C and D were sampled by pipetting 50 μL of each solution into 1 mL of 0.5% modified Griess reagent and vortexed. Absorbance at 540 nm was measured by a spectrophotometer.

It was observed that a healthy human volunteer contains 1-20 ppm of nitrite in saliva. The level of nitrite increased 5-20 folds when dietary nitrate was added. Moreover, no appreciable amount of nitrite was produced without the presence of saliva which contains nitric oxide-producing bacteria.

The top 5 nitrate-rich vegetables were found: beetroot (0.5-1% of nitrate), rocket (0.5%), green bean sprout (0.39-0.45%), kale, spinach, celery (0.24-0.4%). Other vegetables, including lettuce, bok choy, and cabbage, also contained high amounts of nitrate (0.1-0.3%). In addition, cured red meat including hot dog, ham, bacon, etc., contained a high amount of nitrite ranging from 20 ppm to 140 ppm, as KNO₂ or NaNO₂ is often added to meat products for preservation.

### Example 2: Nitrate-Rich Foods for Reducing Oral Malodor and Enhanced Breath Freshening

It was found that in the absence of nitrates, oral bacteria will utilize sulfur-bearing proteins as a primary food source, and will breakdown the proteinaceous foods to cystine and cysteines. Cystine and cysteine are then further broken down by the anaerobic oral bacteria to generate hydrogen sulfide, methyl mercaptan and dimethyl sulfide, i.e., VSCs, and produce malodor. It was further found that, in the presence of nitrate-rich foods, oral bacteria will preferentially utilize nitrate as the food source and generate nitric oxide which is taken up by the human circulation system via sublingual absorption. This leads to the generation of less VSCs that are responsible for oral malodor, as well as potential damage to teeth and soft/hard tissues.

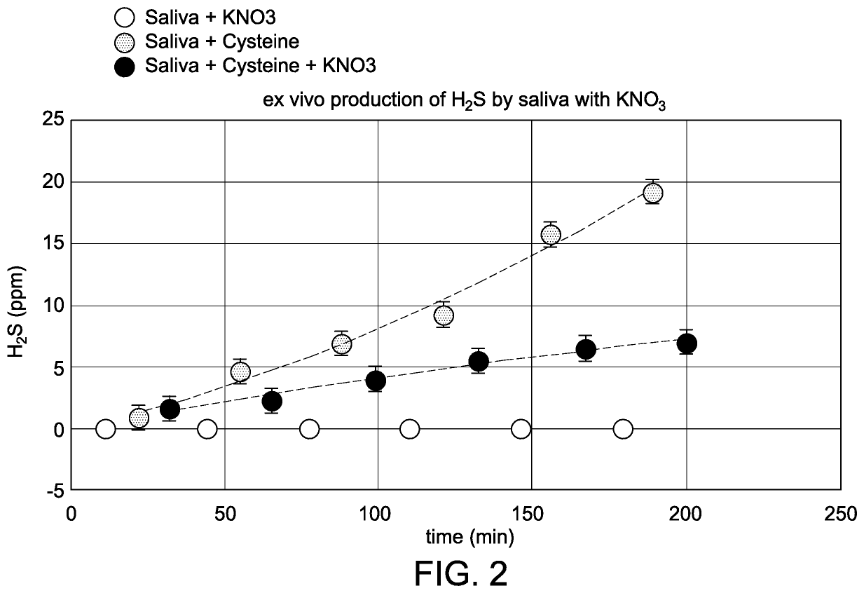

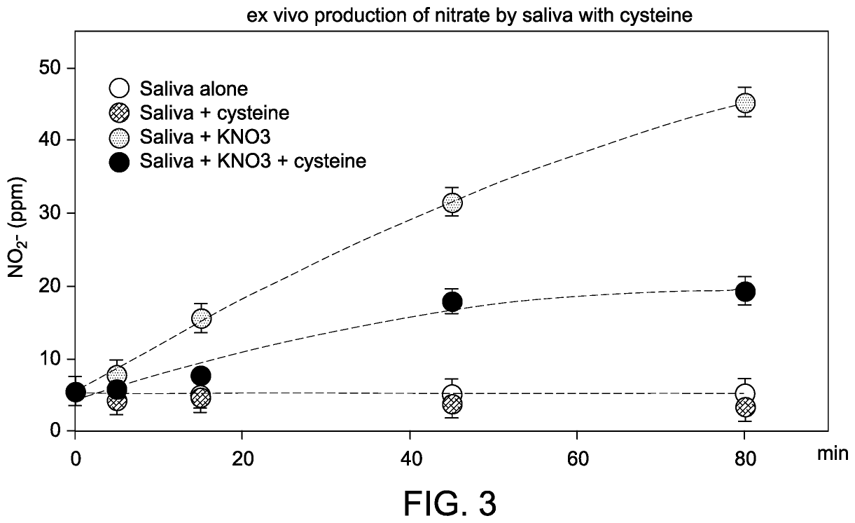

For example, as shown in FIG. 2, when saliva was mixed with cysteine, a sulfur-bearing amino acid, 20 ppm of H₂S was generated in the headspace air tested by a Gas chromatograph equipped with a chemiluminescent sulfur detector. However, when saliva was mixed with cysteine in the presence of potassium nitrate, or beetroot powder, less than 5 ppm of H₂S was generated. Furthermore, as shown in FIG. 3, more than 50 ppm of nitrite was observed in the headspace vial when dietary nitrate was added to the saliva. It was concluded that when dietary nitrate is added into an oral composition, it can significantly reduce volatile sulfuric compounds that are responsible for oral malodor, while converting nitrate to nitrite, which may lead to the benefit of a decreased systolic and diastolic blood pressure and enhanced heart health.

### Example 3: Testing of Nitrate Reductase (NR) Activity of Oral Bacteria

### General Methods

### Test Bacteria, Growth and Nitrate Reductase Assay

Gram negative anaerobic oral bacterial frequently associated with dorsum of the tongue and halitosis were tested. These include: Fusobacterium nucleatum (ATCC 10953). Porphyromonas gingivalis (ATCC 33277), Actinomyces naeslundii AN19 and MG1, Enterococcus faecalis (ATCC29212), Streptococcus gordonii, Streptococcus mutans UA159, Streptococcus sangunii, Streptococcus sobrinus, and Veillonella atypica (ATCC 17744) (V.a.).

For evaluation of nitric oxide producing capability of oral bacteria, overnight cultures of test organisms were washed once and transferred into a medium without any nitrate or nitrite compounds. A 0.5% (or 1%) KNO₃ was added to the medium, and further incubated anaerobically at 37° C. for 24-48 hours. The concentration of nitrite (due to reduction of nitrate by nitrate reductase) was determined by Griess Test over a desired period of time.

### Effect of Natural and Chemical Antimicrobial Agents on Nitrite Production

To examine the effect of natural and chemical antimicrobial agents on NO production, test bacteria were pre-exposed to magnolia bark extract (MBE) (a natural germ-kill compound), peppermint oil, and chlorhexidine (CHX). The activities of nitrate reductase from oral bacteria were assayed by Griess test as described above. No treatment controls were included for each test bacterial species. The production of nitrite was reduced if the test antimicrobial compounds affected the nitrate reductase activity. Dose response curves were prepared.

### Effect of Addition of Cysteine on NR Activity by Saliva Bacteria

Human stimulated saliva was collected from two subjects and tested for NR activity in the presence of nitrate, cysteine and a combination of nitrate and cysteine. The Griess test was used for nitrite production.

### Effect of Nitrate Reductase on Volatile Sulfur Compounds (VSC) Production

Test bacteria A.a. and V.a were incubated with nitrate, cystine or nitrate/cystine, and the production of VSCs were measured using a portable OralChroma. This instrument detects three volatile sulfur compounds. The effect of nitrate reductase activity on in vitro VSC production by test bacteria was investigated.

### Example 4: Nitrate Reductase (NR) Activity of Human Salivary Bacteria

### Methods

### Collection of Stimulated Saliva

Two subjects were evaluated in this study. The subjects refrained from oral hygiene and eating the night before and morning of the test and their stimulated saliva was collected after chewing 1 g of gum base for 5 minutes. The saliva samples were stored immediately on ice, diluted 1:1 with D.I. H₂O and used as a bacterial source for nitrate reductase (NR) activity testing under anaerobic condition.

### Test Protocol

The contents in each reaction mixture contained (mL):

| | KNO₃ | Cysteine | KNO₃/Cys | No cells | Cells only |
|---|---|---|---|---|---|
| Saliva | 0.5 | 0.5 | 0.5 | 0 | 0.5 |
| D.I. water | 0.5 | 0 | 0 | 0.5 | 0.5 |
| Cysteine | 0 | 0.5 | 0.5 | 0.5 | 0 |
| (0.5%) | | | | | |
| KNO₃ (30%) | 0.1 | 0 | 0.1 | 0.1 | 0 |

Detection of the end product of NR and nitrite was by the Griess reagent at OD_{550 nm} readings.

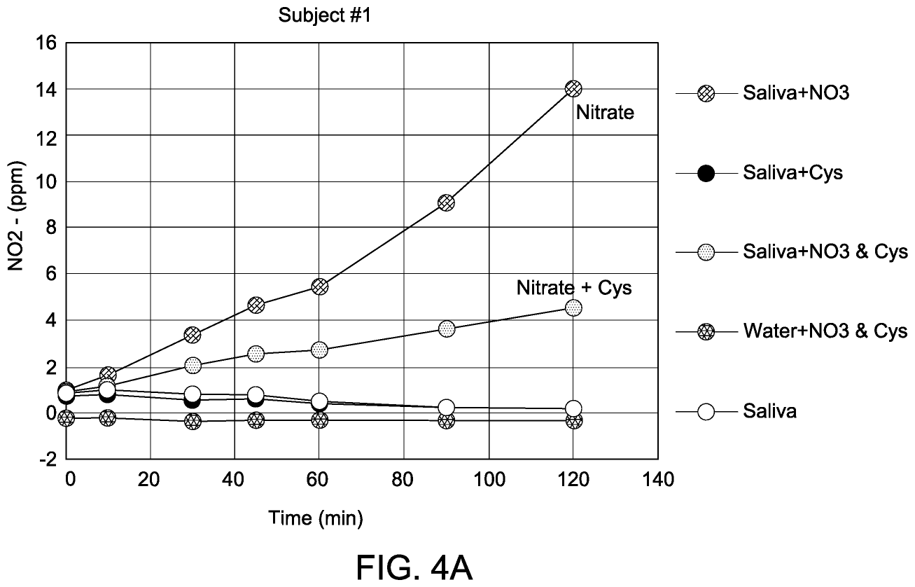

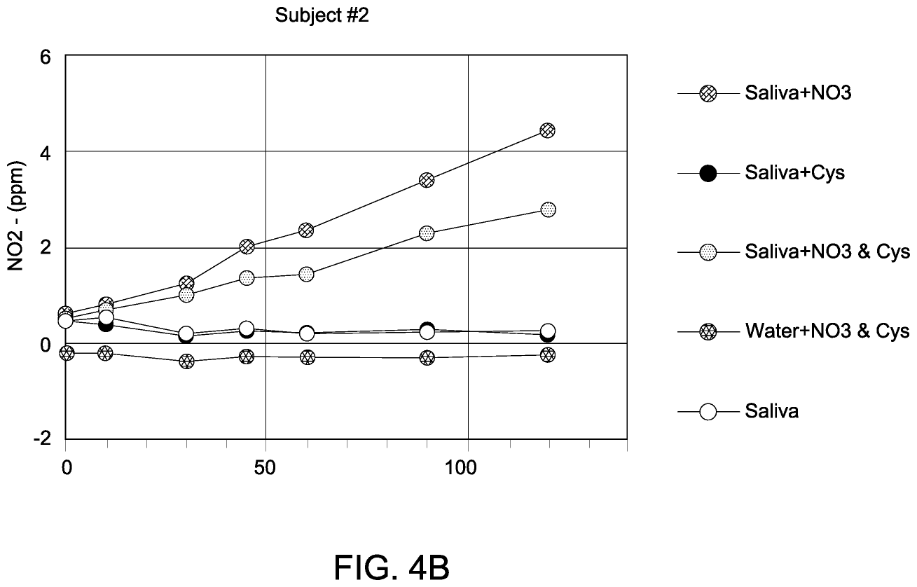

As shown in FIGS. 4A and 4B, saliva samples from both subjects showed similar results, i.e., nitrate (KNO₃, 3%) induced NR activity of salivary bacteria to produce nitrite. When cysteine (0.25%) was present in the assay reaction mixture, in addition to nitrate, a decrease in NR activity was noted.

The same test was further performed on pooled saliva from 4 subjects and adjusted to different concentration (OD_{600 nm}=0.7, 1.0 and 1.5), and it was similarly found that the addition of cysteine reduced 23-49% of NR activity.

### Example 5: Nitrate Reductase (NR) Activity of Selected Test Oral Bacteria

Test bacteria: Actinomyces naeslundii (AN19 and MG1) (A.n.)
Aggregatibacter actinomycetemcomitans (A.a.)
Fusobacterium nucleatum subsp polymorphum (ATCC10593) (F.n.)
Porphyromonas gingivalis (ATCC33277 and w83) (P.g.)
Streptococcus gordonii (S.g.)
Streptococcus mutans (UA159) (S.m.)
Veillonella atypica (ATCC 17744) (V.a)

### Growth Media:

A. actinomycetemcomitans (A.a.) was grown anaerobically in THB broth supplemented with 1% yeast extract with 0.001% Hemin, and 0.0001% Vitamin K.

P. gingivalis and F. nucleatum were grown anaerobically in THB broth supplemented with 0.001% Hemin and 0.0001% Vitamin K.

A. naeslundii, S. gordonii, S. mutans, were grown anaerobically in BHI broth for 24 hours.

V. atypica was grown in RCM medium without (NH₄)₂SO₄ (to avoid the NH₄ affecting Griess reagent); and without agar (0.05%, to avoid affecting the OD measurement).

### NR Activity Assayed in Reaction Mixture Containing KNO₃ in Bacterial Growth Media

The nitrate source KNO₃ (0.1% final) was added to respective growth media for individual test bacteria and incubated for 24-48 hours, then 20 μl of the culture broth was withdrawn and tested for nitrite with Griess reagent.

No NR activity was detected, i.e., no nitrite production, when the KNO₃ source was not present in the growth media with the test bacteria.

Bacteria with positive NR activity included A. actinomycetemcomitans (A.a.), A. naeslundii (A.n.) and V. atypica (V.a.). The NR activity for A.a. was also detected under aerobic incubation. A.a. and V. atypica were selected for further testing.

### NR Activity Using Harvested Cells (24-48 Hrs) of Test Bacteria in a Buffer System (PBS)

Test Bacteria: A. actinomycetemcomitans (A.a.), V. atypica (V.a), and A. naeslundii (AN19 & MG1) (A.n.)

Test bacteria were grown in respective media anaerobically for 48 hours. Cells were centrifuged and re-suspended in buffer or fresh medium for NR activity testing.

A. actinomycetemcomitans (A.a.): The NR activity was detected in both the PBS and growth medium. When tested in PBS, the NR activity was not as stable and reproducible as in medium.

V. atypica (V.a): NR activity was detected in the PBS and reduced transport buffer. The NR activity was not stable and was lower in PBS. Activity was reproducible in reduced transport buffer.

A. naeslundii (AN19 and MG1) (A.n.): Both strains were positive for NR activity. In A. naeslundii AN19, NR could be induced in both PBS and BHI medium. Strain MG1 worked best in BHI medium while little NR was detected in the PBS system.

### Example 6: Effect of Chlorhexidine (CHX) on NR Activity of Test Oral Bacteria

Effect of CHX on NR Activity of A. actinomycetemcomitans (A.a.)

### Short-Term Time-Kill of CHX Against A.a.

Fresh grown cells were harvested and adjusted to OD_{550 nm}=0.5, then mixed with CHX (75-1,000 μg/ml). At different time points, an aliquot of cells was removed and mixed with (or without) stop reagent (0.5% Tween 80 and 0.07% Lecithin) to stop the CHX reaction on cells. The treated cells were centrifuged to remove the supernatant that contained CHX, and the cell pellet was spotted on BHI agar (supplemented with 0.001% Hemin, 0.0001% Vitamin K) for cell viability.

When stop reagent was added to the cells/CHX mixture after treatment, there was limited viability after two-minute treatment with CHX at concentrations ranging from 75-1,000 μg/ml, as shown below in Table 1 (0.1%) (+: viable colony; −: no viable colony).

TABLE 1

| CHX | | Treatment Time (min) | | | | | | | |
|---|---|---|---|---|---|---|---|---|---|
| (μg/ml) | % | 0 | 1 | 2 | 3 | 5 | 10 | 30 | 60 |
| 75 | 0.0075 | + | | | | + | | + | + |
| 100 | 0.01 | + | + | | + | + | + | + | − |
| 200 | 0.02 | + | + | + | + | + | + | − | − |
| 300 | 0.03 | + | + | + | + | + | + | − | − |
| 500 | 0.05 | + | + | + | + | + | − | − | − |
| 750 | 0.075 | + | + | + | + | + | − | − | − |
| 1000 | 0.1 | + | + | + | − | − | | − | − |

### NR Activity of CHX Treated A.a.:

Treatment with 100 μg/ml for 10 min inhibited NR activity
Treatment with 1,000 μg/ml (0.1% present in CHX mouth rinse) for 1 min completely inhibited NR activity of A.a.

These results are shown in Tables 2 and 3 below:

TABLE 2
Values represent OD at 550 nm for A.a. NR activity test

| | 1 min treatment | | | | 10 min treatment | | |
|---|---|---|---|---|---|---|---|
| CHX | 1^{st} trial | 2^{nd} trial | | 3^{rd} trial | 1^{st} trial | | 2^{nd} trial |
| (μg/ml) | 2 hr | 2 hr | 3 hr | 2 hr | 2 hr | 3 hr | 2 hr |
| 0 | 0.837 | 0.807 | 0.839 | 0.853 | 0.841 | 0.813 | 0.853 |
| 100 | | 0.212 | 0.856 | 0.844 | 0.117 | 0.130 | 0.284 |
| 200 | | 0.279 | 0.849 | 0.852 | 0.125 | 0.127 | 0.125 |
| 300 | 0.263 | 0.134 | 0.260 | 0.853 | 0.115 | 0.130 | 0.129 |
| 500 | 0.284 | 0.107 | 0.213 | 0.443 | 0.114 | 0.118 | 0.131 |
| 750 | 0.148 | 0.107 | 0.169 | 0.391 | 0.118 | 0.141 | 0.112 |
| 1000 | 0.160 | | | | | | |

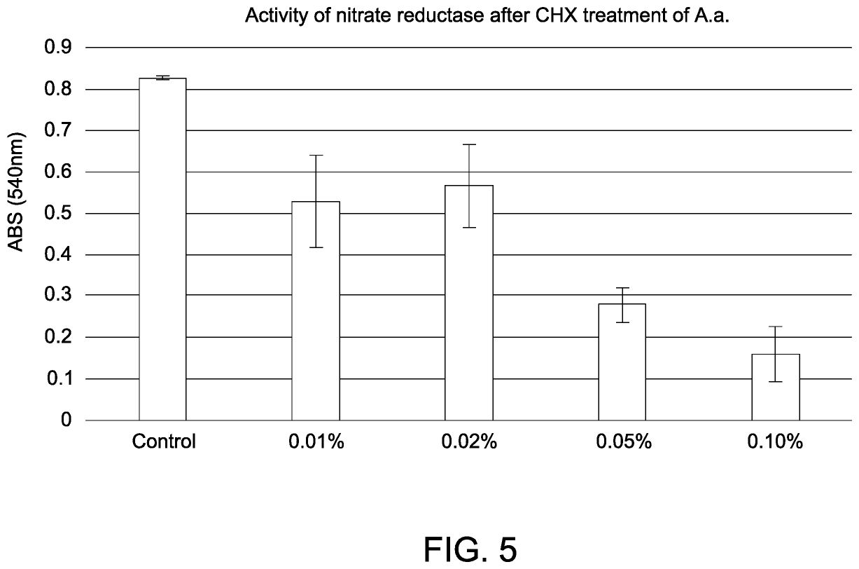

The activity of nitrate reductase after CHX treatment of A.a. is further illustrated in FIG. 5.

### Effect of CHX on NR Activity of V. Atypica (V.a)

### Short-Term Time-Kill of CHX Against V.a

Short-term time-kill experiments of CHX against V.a were performed as described for A.a.

### NR Activity of CHX Treated V.a:

The method used was the same as that for A.a., except the V.a cell concentration was adjusted to OD_{550 nm}=0.7 (10⁸ CFR/ml) in Reduced Transport buffer. The NR activity was also determined in the same buffer system. Results are shown below in Table 3.

TABLE 3

| | 1 min Treatment | 10 min Treatment |
|---|---|---|
| CHX (μg/ml) | OD | OD |
| 0 | 0.325 | 0.315 |
| 10 | 0.266 | 0.300 |
| 50 | 0.304 | 0.282 |
| 100 | 0.255 | 0.280 |
| 250 | 0.190 | 0.161 |
| 500 | 0.170 | 0.162 |

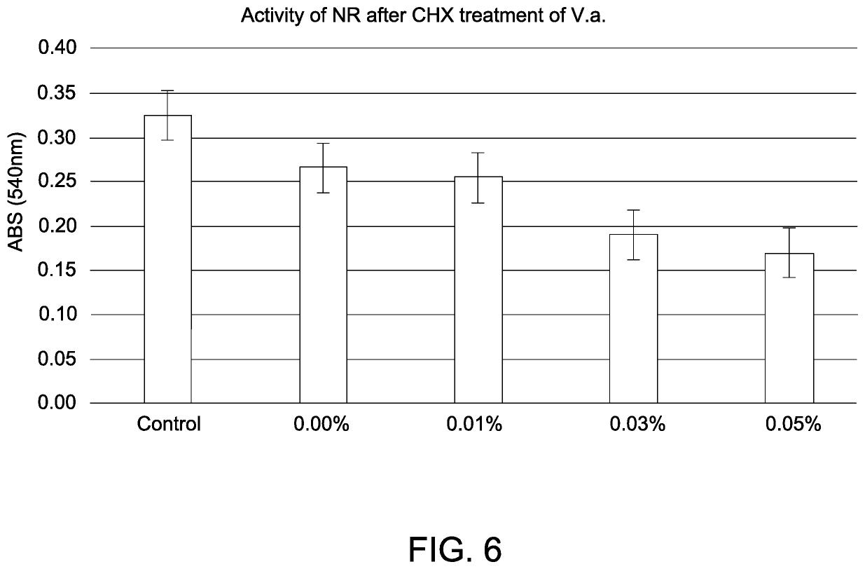

The activity of nitrate reductase after CHX treatment of V.a is further illustrated in FIG. 6.

### Example 7: Effect of Magnolia Bark Extract (MBE) on NR Activity of Test Oral Bacteria

### Effect of MBE on NR Activity of A. Actinomycetemcomitans (A.a)

### Short-Term Time-Kill of MBE Against A.a

Fresh grown cells were harvested and mixed with MBE for a short period of time and the viability of treated cells were evaluated. 10% DMSO was used to wash the treated cells and the MBE was removed from the assay system.

Immediately after treatment, 10% DMSO was added to the cell mixture. The cell mixture was then centrifuged and washed once more with 10% DMSO. Cells treated with MBE for various times were spotted on an agar plate, % DMSO and noted for viable colony development.

As shown in Table 4 below, only a few colonies on agar plates were noted for A.a. treated with 100 and 250 μg/ml MBE. No growth was detected after treatment with MBE at ≥500 μg/ml.

TABLE 4

| MBE (μg/ml) | 0 | 25 | 50 | 75 | 100 | 250 | 500 | 750 | 1000 |
|---|---|---|---|---|---|---|---|---|---|
| Colony | + | + | + | + | + | + | − | − | − |

NR activity of A.a. after treatment with MBE is shown in Tables 5 and 6 below:

TABLE 5
A.a. Treated with MBE 1 min (NR activity at 2 hr or 3 hr)(OD)

| | OD (1 min treatment) | | | | | | | |
|---|---|---|---|---|---|---|---|---|
| | 1^{st} trial | 2^{nd} trial | 3^{rd} trial | | 4^{th} trial | | 5^{th} trial (with | |
| MBE | (EtOH) | (EtOH) | (EtOH) | | (in DMSO) | | stop reagent) | |
| (μg/ml) | 3 hr | 2hr | 2 hr | 3 hr | 2 hr | 3 hr | 2 hr | 3 hr |
| 0 | 0.787 | 1.025 | 0.792 | 0.806 | 0.743 | 0.803 | 0.948 | 1.232 |
| 50 | 0.769 | 1.000 | 0.787 | 0.786 | 0.711 | 0.890 | | |
| 75 | | 0.888 | 0.788 | 0.800 | 0.540 | 0.900 | 0.996 | 1.179 |
| 100 | 0.827 | 1.030 | 0.485 | 0.773 | 0.400 | 0.904 | 1.012 | 1.195 |
| 150 | | | 0.518 | 0.797 | 0.189 | 0.767 | | |
| 200 | | 0.914 | 0.117 | 0.193 | 0.122 | 0.361 | 1.030 | 1.209 |
| 300 | 0.110 | | 0.103 | 0.095 | 0.124 | 0.115 | | |

TABLE 6
A.a. treated with MBE 10 min (NR activity at 2 hr or 3 hr)

| | OD (10 min treatment) | | | | | | | | |
|---|---|---|---|---|---|---|---|---|---|
| MBE | 1^{st} trial | 2^{nd} trial | | 3^{rd} trial | | 4^{th} trial | | 5^{th} trial (with | |
| (μg/ | (EtOH) | (EtOH) | | (EtOH) | | (in DMSO) | | stop reagent) | |
| ml) | 3 hr | 2 hr | 3 hr | 2 hr | 3 hr | 2 hr | 3 hr | 2 hr | 3 hr |
| 0 | 0.872 | 0.583 | 0.982 | 0.790 | 0.841 | 0.841 | 0.958 | 0.867 | 1.212 |
| 10 | 0.811 | | | | | | | | |
| 50 | 0.814 | 0.621 | 0.968 | 0.799 | 0.836 | 0.828 | 0.980 | | |
| 75 | | 0.741 | 0.981 | 0.323 | 0.795 | 0.174 | 0.893 | 0.707 | 1.195 |
| 100 | 0.102 | 0.477 | 0.806 | 0.105 | 0.123 | 0.092 | 0.163 | 0.395 | 1.069 |
| 150 | | | | 0.104 | 0.104 | 0.089 | 0.133 | | |
| 200 | | 0.118 | 0.114 | 0.103 | 0.095 | 0.106 | 0.133 | 0.166 | 0.146 |
| 250 | 0.136 | | | | | | | | |
| 300 | | | | 0.112 | 0.101 | 0.134 | 0.119 | | |

After exposure for 1 min with 100 μg/ml of MBE, no changes in NR activity of A.a. was observed. At 50 μg/ml 10 min treatment time, >90% NR activity still remained. Longer treatment time of 10 min at 100 μg/ml inhibited the NR activity.

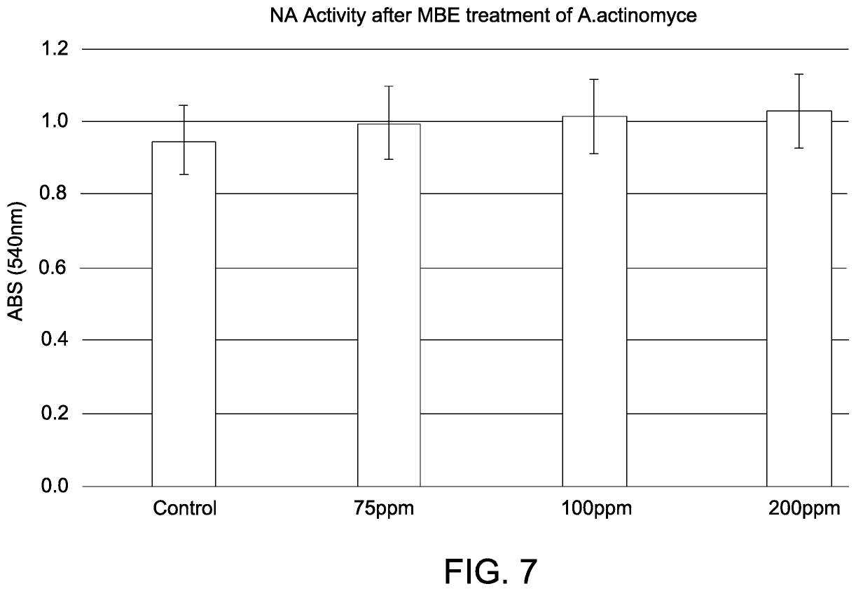

The activity of nitrate reductase after MBE treatment of A.a is further illustrated in FIG. 7.

Effect of MBE on NR Activity of V. atypica (V.a)

Before MBE on NR activity was tested, its MIC and short-term kill concentration against A.a was first determined. MBE was dissolved in 100% EtOH. The MIC of MBE for V.a was 25 μg/ml.

### Short-Term Time-Kill of MBE Against V.a

It was discovered that V.a requires the use of a special transfer buffer instead of PBS for assay system for viability.

The NR activity of V. atypica after treatment with MBE is shown below in Tables 7 and 8.

TABLE 7
V.a treated with MBE 1 min (NR activity at 2 hr or 3 hr)(OD)

| | OD (V.a 1 min treatment) | | | | | | | |
|---|---|---|---|---|---|---|---|---|
| | 1^{st} trial | 2^{nd} trial | 3^{rd} trial | | 4^{th} trial (with | | 5^{th} trial (with | |
| MBE | (EtOH) | (EtOH) | (EtOH) | | stop reagent) | | stop reagent) | |
| (μg/ml) | 3 hr | 2 hr | 2 hr | 3 hr | 2 hr | 3 hr | 2 hr | 3 hr |
| 0 | 0.254 | 0.169 | 0.212 | 0.345 | 0.332 | 0.552 | 0.373 | 0.546 |
| 10 | 0.313 | | | | | | | |
| 25 | 0.314 | 0.170 | | | | | | |
| 50 | 0.303 | 0.186 | 0.172 | 0.192 | 0.292 | 0.368 | 0.339 | 0.500 |
| 75 | | 0.199 | 0.458 | 0.177 | 0.301 | 0.334 | 0.360 | 0.490 |
| 100 | | 0.200 | 0.161 | 0.179 | 0.259 | 0.287 | 0.341 | 0.455 |
| 150 | | | | | 0.188 | 0.182 | 0.334 | 0.393 |
| 200 | | | 0.121 | 0.126 | 0.120 | 0.104 | 0.239 | 0.216 |
| 300 | | | | | 0.105 | 0.081 | 0.178 | 0.147 |

TABLE 8
V.a treated with MBE 10 min (NR activity at 2 hr or 3 hr)

| | OD (V.a 10 min treatment) | | | | | | | | | |
|---|---|---|---|---|---|---|---|---|---|---|
| | 1^{st} trial | 2^{nd} trial | 3^{rd} trial | | 4^{th} trial | | 5^{th} trial (with | | 6^{th} Trial (with | |
| MBE | (EtOH) | (EtOH) | (EtOH) | | (EtOH) | | stop reagent) | | stop reagent) | |
| (μg/ml) | 3 hr | 2 hr | 2 hr | 3 hr | 2 hr | 3 hr | 2 hr | 3 hr | 2 hr | 3 hr |
| 0 | 0.209 | 0.170 | 0.274 | 0.283 | 0.272 | 0.433 | 0.176 | 0.206 | 0.307 | 0.397 |
| 10 | 0.233 | | | | | | | | | |
| 25 | 0.303 | 0.200 | | | | | | | | |
| 50 | 0.302 | 0.264 | 0.183 | 0.183 | 0.280 | 0.307 | 0.276 | 0.299 | 0.335 | 0.377 |
| 75 | | 0.246 | 0.188 | 0.189 | 0.255 | 0.279 | 0.293 | 0.303 | 0.347 | 0.353 |
| 100 | | 0.247 | 0.173 | 0.151 | 0.193 | 0.192 | 0.258 | 0.262 | 0.307 | 0.318 |
| 150 | | | | | 0.138 | 0.130 | 0.062 | 0.058 | 0.182 | 0.161 |
| 200 | | | 0.152 | 0.134 | 0.084 | 0.068 | 0.058 | 0.050 | 0.148 | 0.122 |
| 300 | | | | | 0.100 | 0.076 | 0.078 | 0.063 | 0.171 | 0.141 |

At 50-75 μg/ml of MBE for 1 min treatment with stop reagent, less than 10% inhibition of NR activity was noted. Similarly, treatment of V.a with MBE at 50 μg/ml for 10 min did not show significant NR activity inhibition.

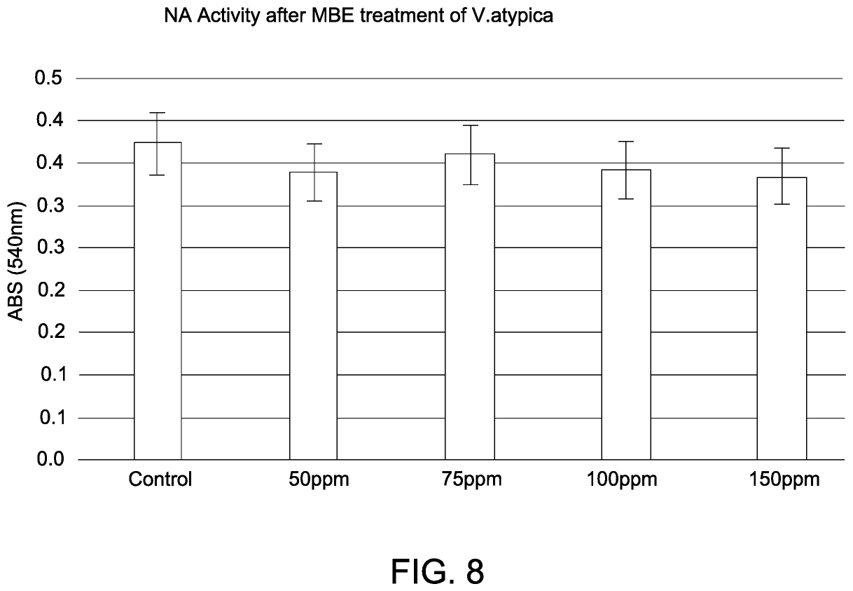

The activity of nitrate reductase after MBE treatment of V.a is further illustrated in FIG. 8.

### Example 8: The Effect of Peppermint on NR Activity of A.a. and V.a

MICs of peppermint oil for A.a., P. gingivalis and F. nucleatum were >5 μl/ml. MIC for V.a. was 1.25 μl/ml.

### Effect of Peppermint Oil on NR Activity of A.a

Test bacteria cells were incubated with peppermint oil (1 μl/ml and 10 μl/ml concentration prepared from stock sample) in growth medium and 0.3% of nitrate source. NR activity was determined up to 3 hours and the results show in Table 9 below. Controls contained no peppermint oil.

TABLE 9

| | Peppermint treatment (1 μl/ml) | |
|---|---|---|
| NR activity | 1^{st} trial | 2^{nd} trial |

| (OD) | Control | Test | Control | Test |
|---|---|---|---|---|
| 2 hr | 0.314 | 0.250 | 1.051 | 1.029 |
| 3 hr | 0.908 | 0.841 | 0.980 | 0.983 |

The NR activity of A.a, when incubated in the presence of 1 μl/ml or 1000 ppm of peppermint oil and nitrate, was not affected.

### Effect of Peppermint Oil on NR Activity of V.a

TABLE 10

| | Peppermint treatment (1 μl/ml) | |
|---|---|---|
| NR activity | 1^{st} trial | 2^{nd} trial |

| (OD) | Control | Test | Control | Test |
|---|---|---|---|---|
| 2 hr | 0.665 | 0.484 | 0.887 | 0.479 |
| 3 hr | 1.024 | 0.910 | 1.102 | 0.920 |

1 μl/ml of peppermint inhibited about 20% of V. atypica NR activity after 3 hr of incubation.

### Example 9: The Effect of Cysteine on NR Activity of Test Bacteria

### Effect of Cysteine on NR Activity of A.a

Test bacteria cells were incubated with 1% cysteine in growth medium and 0.3% of a nitrate source. NR activity was determined up to 3 hours and the results shown in Tables 18 and 19. Controls contained no cysteine.

TABLE 11
NR activity of A.a in the presence of cysteine

| | A.a - Cysteine (1%) | | | | | |
|---|---|---|---|---|---|---|
| NR activity | 1^{st} trial | | 2^{nd} trial | | 3^{rd} trial | |
| (OD) | Control | Test | Control | Test | Control | Test |
| 2 hr | 0.314 | 0.207 | 1.051 | 0.994 | 1.022 | 0.832 |
| 3 hr | 0.908 | 0.672 | 0.980 | 0.928 | 1.033 | 0.919 |

NR activity of A.a was not significantly affected by the addition of cysteine.

### Effect of Cysteine on NR Activity of V.a

TABLE 12
NR activity of V.a in the presence of cysteine

| | A.a - Cysteine (1%) | | | | | |
|---|---|---|---|---|---|---|
| NR activity | 1^{st} trial | | 2^{nd} trial | | 3^{rd} trial | |
| (OD) | Control | Test | Control | Test | Control | Test |
| 2 hr | 0.665 | 0.372 | 0.887 | 0.487 | 1.160 | 1.139 |
| 3 hr | 1.024 | 0.711 | 1.102 | 0.808 | 1.160 | 1.144 |

Results from these examples demonstrated that salivary bacteria, specifically, A. actinomycetemcomitans, A. naeslundii and V. atypica were the major oral bacteria that produce nitrite. Streptococcal species did not contribute significantly to salivary nitrite generation. Treatment of A. actinomycetemcomitans and V. atypica with the natural germ-kill MBE (50-150 μg/ml) or peppermint flavor oils at the level typically incorporated in chewing gum and mints do not significantly affect the activities of nitrate reductase, hence the production of nitrite and impact to cardiovascular risk for these products. On the other hand, treatment of these oral bacteria by chlorhexidine (>=10 μg/ml) significantly inhibits the activities of nitrate reductase from oral bacteria, hence increases the risk for cardiovascular diseases.

### Example 10: Sugar-Free Chewing Gum with Dietary Nitrates

### Sugar-Free Chewing Gums A and B

For each of Gums A and B, gum base was heated to 165° F. and placed in a mixer. High intensity sweeteners, flavors and other ingredients were added to the mixer, followed by dietary nitrates. The mixture was mixed for 7-12 minutes to ensure uniformity. The mixture was then sheeted and cut, with a piece weight of 2.75 g. The composition of Gums A and B are shown below in Table 13.

TABLE 13

| | Sugar Free Gum A | | Sugar Free Gum B | |
|---|---|---|---|---|
| | Percentage | Weight | Percentage | Weight |
| Compound | % | (g) | % | (g) |
| Sorbitol | 69.1 | 691 | 63.512 | 635.12 |
| Gumbase | 27.47 | 274.7 | 27.47 | 274.7 |
| Flavor | 2.515 | 25.15 | 2.515 | 25.15 |
| Aspartame | 0.345 | 3.45 | 0.345 | 3.45 |
| Acesulfame K | 0.3 | 3 | 0.938 | 9.38 |
| Acesulfame K | 0.059 | 0.59 | 0.059 | 0.59 |
| Acacia | 0.151 | 1.51 | 0.151 | 1.51 |
| KNO₃ | 0.05 | 0.5 | | |
| Beet root powder | | | 5 | 50 |
| Colorant | 0.01 | 0.1 | 0.01 | 0.1 |
| Total | 100.0 | 1000 | 100.0 | 1000 |

### Example 11: Dual-Layer Compressed Mints with Dietary Nitrates

### Dual-Layer Mints A and B

For each of dual-layer Mints A and B, all ingredients were mixed using a Hobart mixer. 0.5 g of each layer was charged to a compressor. The layers were compressed to form dual-layer compressed mints. The hardness was adjusted to 15-18 kPa, and piece weight adjusted to 1.0 g. The composition of dual-layered Mints A and B are shown below in Table 14.

| | Dual Layer Strawberry Mint A | | | | Dual Layer Raspberry Mint B | | | |
|---|---|---|---|---|---|---|---|---|
| | Strawberry Mint | | Strawberry Mint | | Raspberry Mint | | Raspberry Mint | |
| | (Colored Layer) | | (White Layer) | | (Colored Layer) | | (White Layer) | |
| Ingredient | % | Grams | % | Grams | % | Grams | % | Grams |
| Sorbitol | 92.103 | 460.52 | 94.675 | 473.38 | 90.950 | 454.75 | 94.052 | 470.26 |
| Aspartame | 0.120 | 0.60 | 0.120 | 0.60 | 0.120 | 0.60 | 0.120 | 0.6 |
| Sucralose | 0.040 | 0.20 | 0.040 | 0.20 | 0.020 | 0.10 | 0.020 | 0.10 |
| Acesulfame K | 0.120 | 0.60 | 0.120 | 0.60 | 0.030 | 0.15 | 0.030 | 0.15 |
| Strawberry Flavor | 1.67 | 8.35 | 1.67 | 8.35 | | 0.00 | | 0.00 |
| Raspberry Flavor | | 0.00 | | 0.00 | 2.93 | 14.67 | 2.93 | 14.67 |
| Citric Acid | | 0.00 | 0.53 | 2.65 | | 0.00 | 1.25 | 6.25 |
| Malic Acid | | 0.00 | 2.00 | 10.00 | | 0.00 | 0.75 | 3.75 |
| Colorant | 0.052 | 0.26 | | 0.00 | 0.052 | 0.26 | | 0.00 |
| KNO₃ | 0.050 | 0.25 | | 0.00 | 0.05 | 0.25 | | 0.00 |
| Beet Root Powder | 5.000 | 25.00 | | 0.00 | 5.00 | 25.00 | | 0.00 |
| High intensity coolant | 0.040 | 0.20 | 0.040 | 0.20 | 0.040 | 0.20 | 0.040 | 0.20 |
| Menthol | 0.005 | 0.03 | 0.005 | 0.03 | 0.005 | 0.03 | 0.005 | 0.03 |
| Magnesium Stearate | 0.80 | 4.00 | 0.80 | 4.00 | 0.80 | 4.00 | 0.80 | 4.00 |
| Total | 100.00 | 500.00 | 100.00 | 500.00 | 100.00 | 500.00 | 100.00 | 500.00 |

This written description uses examples to disclose the technology, including the best mode, and also to enable any person skilled in the art to practice the technology, including making and using any devices or systems and performing any incorporated methods. The patentable scope of the technology is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1.-33. (canceled)

34. An oral composition comprising an effective amount of a nitrate for reducing volatile sulfur compounds in the oral cavity; wherein the nitrate comprises a combination of:
a sodium nitrate or potassium nitrate; and
a nitrate-rich food.

35. The oral composition of claim 34, wherein the nitrate further comprises a nitrate-rich extract.

36. The oral composition of claim 34, wherein the nitrate is present in an amount of about 0.01% to about 5% by weight of the oral composition.

37. The oral composition of claim 34, wherein the nitrate-rich food is selected from beetroot, kale, arugula, chard, spinach, parsley, watercress, fennel, Chinese cabbage, bok choy, leek, lettuce, celery, radish, turnip, rocket, beet greens, mustard greens, carrots, onions, garlic, kohlrabi, chicory leaf, bean sprout, watermelon, kiwi, apples, pomegranate, bananas, oranges, strawberries, peaches, pears, grapes, chocolate, or combinations thereof.

38. The oral composition of claim 35, wherein the nitrate-rich extract is selected from beetroot extract, celery extract, or combinations thereof.

39. The oral composition of claim 34, wherein the composition is in the form of a confectionary composition selected from a breath mint, low boiled candy, chewing gum, chewy candy, hard boiled candy, coated candy, lozenge, syrup, pressed mint, throat drop, or chocolate.

40. The oral composition of claim 39, wherein the chewing gum is a sugar-free chewing gum.

41. An oral composition comprising
an effective amount of a nitrate for reducing volatile sulfur compounds in the oral cavity; wherein the nitrate comprises a combination of:
a sodium nitrate or potassium nitrate; and
a nitrate-rich food; and
an effective amount of ascorbic acid, or a suitable salt thereof.

42. The oral composition of claim 41, wherein the nitrate further comprises a nitrate-rich extract.

43. The oral composition of claim 41, wherein the nitrate is present in an amount of about 0.01% to about 5% by weight of the oral composition.

44. The oral composition of claim 41, wherein the ascorbic acid, or a suitable salt thereof, is present in an amount of about 0.01% to about 5% by weight of the oral composition.

45. The oral composition of claim 41, wherein the nitrate-rich food is selected from beetroot, kale, arugula, chard, spinach, parsley, watercress, fennel, Chinese cabbage, bok choy, leek, lettuce, celery, radish, turnip, rocket, beet greens, mustard greens, carrots, onions, garlic, kohlrabi, chicory leaf, bean sprout, watermelon, kiwi, apples, pomegranate, bananas, oranges, strawberries, peaches, pears, grapes, chocolate, or combinations thereof.

46. The oral composition of claim 42, wherein the nitrate-rich extract is selected from beetroot extract, celery extract, or combinations thereof.

47. The oral composition of claim 41, wherein the composition is in the form of a confectionary composition selected from a breath mint, low boiled candy, chewing gum, chewy candy, hard boiled candy, coated candy, lozenge, syrup, pressed mint, throat drop, or chocolate.

48. The oral composition of claim 47, wherein the chewing gum is a sugar-free chewing gum.

49. A confectionary composition comprising an effective amount of a nitrate for reducing volatile sulfur compounds in the oral cavity; wherein the nitrate comprises a combination of:
a sodium nitrate or potassium nitrate; and
a nitrate-rich food.

50. The confectionary composition of claim 49, wherein the nitrate further comprises a nitrate-rich extract.

51. The confectionary composition of claim 49, wherein the nitrate is present in an amount of about 0.01% to about 5% by weight of the confectionary composition.

52. The confectionary composition of claim 49, wherein the nitrate-rich food is selected from beetroot, kale, arugula, chard, spinach, parsley, watercress, fennel, Chinese cabbage, bok choy, leek, lettuce, celery, radish, turnip, rocket, beet greens, mustard greens, carrots, onions, garlic, kohlrabi, chicory leaf, bean sprout, watermelon, kiwi, apples, pomegranate, bananas, oranges, strawberries, peaches, pears, grapes, chocolate, or combinations thereof.

53. The confectionary composition of claim 50, wherein the nitrate-rich extract is selected from beetroot extract, celery extract, or combinations thereof.

54. The confectionary composition of claim 49, wherein the confectionary composition is in the form of a breath mint, low boiled candy, chewing gum, chewy candy, hard boiled candy, coated candy, lozenge, syrup, pressed mint, throat drop, or chocolate.

55. The confectionary composition of claim 54, wherein the chewing gum is a sugar-free chewing gum.

56. The confectionary composition of claim 49, wherein the composition further comprises ascorbic acid, or a suitable salt thereof.

57. The confectionary composition of claim 56, wherein the ascorbic acid, or a suitable salt thereof, is present in an amount of about 0.01% to about 5% by weight of the confectionary composition.

58. A method for freshening breath, the method comprising consuming a confectionary composition comprising an effective amount of a nitrate; wherein the nitrate comprises a combination of:
a sodium nitrate or potassium nitrate; and
a nitrate-rich food.

59. The method of claim 58, wherein the nitrate further a nitrate-rich extract.

60. The method of claim 58, wherein the nitrate is present in an amount of about 0.01% to about 5% by weight of the confectionary composition.

61. The method of claim 58, wherein the nitrate-rich food is selected from beetroot, kale, arugula, chard, spinach, parsley, watercress, fennel, Chinese cabbage, bok choy, leek, lettuce, celery, radish, turnip, rocket, beet greens, mustard greens, carrots, onions, garlic, kohlrabi, chicory leaf, bean sprout, watermelon, kiwi, apples, pomegranate, bananas, oranges, strawberries, peaches, pears, grapes, chocolate, or combinations thereof.

62. The method of claim 59, wherein the nitrate-rich extract is selected from beetroot extract, celery extract, or combinations thereof.

63. The method of claim 58, wherein the confectionary composition is in the form of a breath mint, low boiled candy, chewing gum, chewy candy, hard boiled candy, coated candy, lozenge, syrup, pressed mint, throat drop, or chocolate.

64. The method of claim 63, wherein the chewing gum is a sugar-free chewing gum.

65. The method of claim 58, wherein the confectionary composition further comprises ascorbic acid, or a suitable salt thereof.

66. The method of claim 65, wherein the ascorbic acid, or a suitable salt thereof, is present in an amount of about 0.01% to about 5% by weight of the confectionary composition.
